(19) United States
(12) Patent Application Publication
(10) Pub. No.: US 2015/0328594 A1
(43) Pub. Date: Nov. 19, 2015

(54) POLYIMIDE MEMBRANES WITH VERY HIGH SEPARATION PERFORMANCE FOR OLEFIN/PARAFFIN SEPARATIONS

(71) Applicant: UOP LLC, Des Plaines, IL (US)

(72) Inventors: Carl W. Liskey, Chicago, IL (US); Chunqing Liu, Arlington Heights, IL (US); Howie Q. Tran, Skokie, IL (US)

(73) Assignee: UOP LLC, Des Plaines, IL (US)

(21) Appl. No.: 14/277,247

(22) Filed: May 14, 2014

(51) Int. Cl. B01D 71/64; C08G 75/20; C07C 7/144; B01D 53/22

(52) U.S. Cl. CPC B01D 71/64; B01D 53/22; B01D 53/228; C08G 75/20; C07C 7/144; B01D 2053/221; B01D 2053/224

(57) ABSTRACT

A copolyimide membrane is provided by the present invention that is effective in separating olefins and paraffins. The membrane with very high selectivity and permeability in the present invention is used in a process for separating olefins from a mixture of olefins and paraffins, the process comprising providing a copolyimide membrane with very high selectivity and high permeability described in the present invention which is permeable to said olefin; (b) contacting the olefin/paraffin mixture on one side of the copolyimide membrane with very high selectivity and high permeability described in the present invention to cause the olefin to permeate the membrane; and (c) removing from the opposite side of the membrane a permeate gas composition comprising a portion of the olefin which permeated through the membrane. Ethylene, propylene, butene, or pentene is separated from ethane, propane, butane, or pentane, respectively with up to 99 mole % olefin content in the permeate.

### BACKGROUND OF THE INVENTION

This invention relates to high performance copolyimide membranes with very high selectivity and high permeability for olefin/paraffin separations.

In the past 30-35 years, the state of the art of polymer membrane-based gas separation processes has evolved rapidly. Membrane-based technologies have advantages of both low capital cost and high-energy efficiency compared to conventional separation methods. Membrane gas separation is of special interest to petroleum producers and refiners, chemical companies, and industrial gas suppliers. Several applications of membrane gas separation have achieved commercial success, including nitrogen enrichment from air, carbon dioxide removal from natural gas and from enhanced oil recovery, and also in hydrogen removal from nitrogen, methane, and argon in ammonia purge gas streams. For example, UOP's Separex™ cellulose acetate spiral wound polymeric membrane is currently an international market leader for carbon dioxide removal from natural gas.

Polymers provide a range of properties including low cost, permeability, mechanical stability, and ease of processability that are important for gas separation. Glassy polymers (i.e., polymers at temperatures below their T_{g}) have stiffer polymer backbones and therefore let smaller molecules such as hydrogen and helium pass through more quickly, while larger molecules such as hydrocarbons pass through more slowly as compared to polymers with less stiff backbones. Cellulose acetate (CA) glassy polymer membranes are used extensively in gas separation. Currently, such CA membranes are used for natural gas upgrading, including the removal of carbon dioxide. Although CA membranes have many advantages, they are limited in a number of properties including selectivity, permeability, and in chemical, thermal, and mechanical stability.

The membranes most commonly used in commercial gas and liquid separation applications are asymmetric polymeric membranes and have a thin nonporous selective skin layer that performs the separation. Separation is based on a solution-diffusion mechanism. This mechanism involves molecular-scale interactions of the permeating gas with the membrane polymer. The mechanism assumes that in a membrane having two opposing surfaces, each component is sorbed by the membrane at one surface, transported by a gas concentration gradient, and desorbed at the opposing surface. According to this solution-diffusion model, the membrane performance in separating a given pair of gases (e.g., CO₂/CH₄, O₂/N₂, H₂/CH₄) is determined by two parameters: the permeability coefficient (abbreviated hereinafter as permeability or P_{A}) and the selectivity (α_{A/B}). The P_{A} is the product of the gas flux and the selective skin layer thickness of the membrane, divided by the pressure difference across the membrane. The α_{A/B} is the ratio of the permeability coefficients of the two gases (α_{A/B}=P_{A}/P_{B}) where P_{A} is the permeability of the more permeable gas and P_{B} is the permeability of the less permeable gas. Gases can have high permeability coefficients because of a high solubility coefficient, a high diffusion coefficient, or because both coefficients are high. In general, the diffusion coefficient decreases while the solubility coefficient increases with an increase in the molecular size of the gas. In high performance polymer membranes, both high permeability and selectivity are desirable because higher permeability decreases the size of the membrane area required to treat a given volume of gas, thereby decreasing capital cost of membrane units, and because higher selectivity results in a higher purity product gas.

Light olefins, such as propylene and ethylene, are produced as co-products from a variety of feedstocks in a number of different processes in the chemical, petrochemical, and petroleum refining industries. Various petrochemical streams contain olefins and other saturated hydrocarbons. Typically, these streams are from stream cracking units (ethylene production), catalytic cracking units (motor gasoline production), or the dehydrogenation of paraffins.

Currently, the separation of olefin and paraffin components is performed by cryogenic distillation, which is expensive and energy intensive due to the low relative volatilities of the components. Large capital expense and energy costs have created incentives for extensive research in this area of separations, and low energy-intensive membrane separations have been considered as an attractive alternative.

In principle, membrane-based technologies have advantages of both low capital cost and high-energy efficiency compared to conventional separation methods for olefin/paraffin separations, such as propylene/propane and ethylene/ethane separations. Three main types of membranes have been reported for olefin/paraffin separations. These are facilitated transport membranes, polymer membranes, and inorganic membranes. Facilitated transport membranes, or ion exchange membranes, which sometimes use silver ions as a complexing agent, have very high olefin/paraffin separation selectivity. However, poor chemical stability due to carrier poisoning, high cost, and low flux currently limit practical applications of facilitated transport membranes.

Separation of olefins from paraffins via conventional polymer membranes has not been commercially successful due to inadequate selectivities and permeabilities of the polymer membrane materials, as well as due to plasticization issues. Polymers that are more permeable are generally less selective than are less permeable polymers. A general trade-off has existed between permeability and selectivity (the so-called “polymer upper bound limit”) for all kinds of separations, including olefin/paraffin separations. In recent years, substantial research effort has been directed to overcoming the limits imposed by this upper bound. Various polymers and techniques have been used, but without much success in terms of improving the membrane selectivity. On the other hand, inorganic membranes, such as carbon molecular sieve and zeolite inorganic membranes, potentially offer adequate selectivities. However, they are brittle and currently too costly to be commercially useful for large scale applications.

Accordingly, it is desirable to provide processes for olefin/paraffin separation using cost effective membranes that have high selectivity and that are highly permeable.

The present invention discloses polyimide membranes with unusually high selectivity and permeability for olefin/paraffin separations such as for propylene/propane separation and methods for making and using these membranes.

### SUMMARY OF THE INVENTION

The invention involves a membrane comprising a plurality of repeating units of formula (I); wherein formula (I) is represented by repeating units of

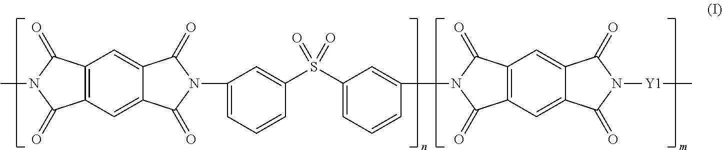

wherein n and m are independent integers from 10 to 500; wherein n/m is in a range of 10:1 to 1:10; wherein Y1 is selected from a group consisting of

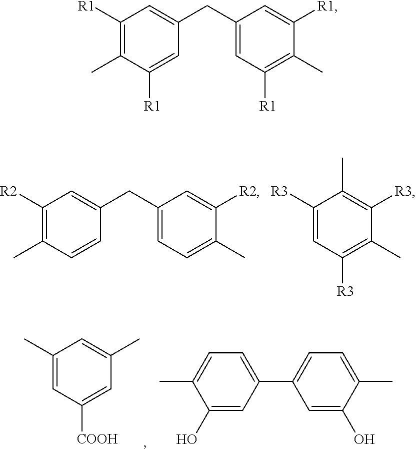

and mixtures thereof, R1 is —(CH₂)ₚCH₃, p is an integer from 0 to 11; R2 is —(CH₂)_{q}CH₃; wherein q is an integer from 0 to 11; and R3 is —(CH₂)ᵣCH₃, r is an integer from 0 to 11.

The polyimide membrane may comprise a poly(3,3′-diaminodiphenyl sulfone-3,3′,5,5′-tetramethyl-4,4′-methylene dianiline-pyromellitic dianhydride). The membrane may be in a form of hollow fibers or tubes, flat sheets, spiral wound modules or corrugated sheets.

Another aspect of the invention is a process of preparing a poly(3,3′-diaminodiphenyl sulfone-3,3′,5,5′-tetramethyl-4,4′-methylene dianiline-pyromellitic dianhydride) membrane comprising reacting 3,3′-diaminodiphenyl sulfone and 3,3′,5,5′-tetramethyl-4,4′-methylene dianiline with pyromellitic dianhydride. The 3,3′-diaminodiphenyl sulfone and 3,3′,5,5′-tetramethyl-4,4′-methylene dianiline may be present at a molar ratio in a range from about 10:1 to 1:10. Preferably, the 3,3′-diaminodiphenyl sulfone and 3,3′,5,5′-tetramethyl-4,4′-methylene dianiline are present at a molar ratio in a range from about 5:1 to 1:5.

The invention further comprises a process for separating olefins from a mixture of olefins and paraffins comprising: (a) providing a polyimide membrane comprising a plurality of repeating units of formula (I).

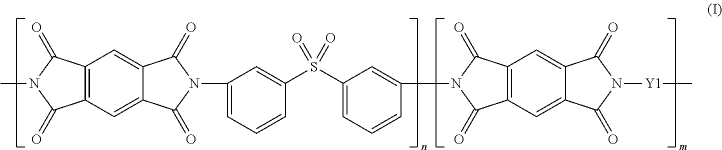

wherein m and n are independent integers from 10 to 500; wherein n/m is in a range of 10:1 to 1:10; wherein Y1 is selected from a group consisting of

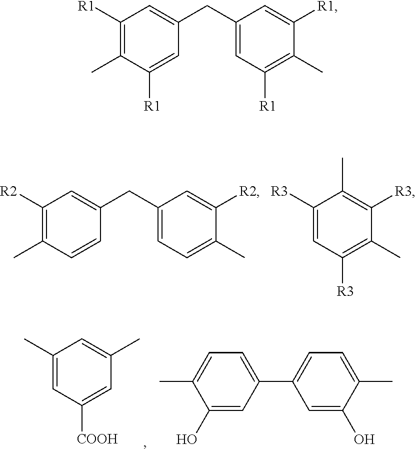

and mixtures thereof; wherein R1 is —(CH₂)ₚCH₃; wherein p is an integer from 0 to 11; wherein R2 is —(CH₂)_{q}CH₃; wherein q is an integer from 0 to 11; and wherein R3 is —(CH₂)ᵣCH₃; wherein r is an integer from 0 to 11; (b) contacting the olefin/paraffin mixture on one side of the polyimide membrane to cause a portion of the olefins to permeate the membrane; and (c) removing from the opposite side of the membrane a permeate gas composition comprising the portion of the olefins which permeated through the membrane.

The olefins may comprise ethylene, propylene, butene, or pentene and the paraffins may comprise ethane, propane, butane, or pentane. The mixture of olefins and paraffins may comprise from about 5 to 95 mass % olefin and about 5 to 95 mass % paraffins. The portion of the olefins that permeated through the membrane may comprise at least 80 mass % olefins. The portion of the olefins which permeated through the membrane may comprise a higher concentration of said olefins than said mixture of olefins and paraffins and the retentate that has not penetrated said membrane may comprise a higher concentration of said paraffins than said mixture of olefins and paraffins.

### BRIEF DESCRIPTION OF THE DRAWINGS

FIG. 1 is a spiral wound membrane module comprising polyimide membranes of the present invention.

FIG. 2 is a hollow fiber membrane module comprising polyimide membranes of the present invention.

FIG. 3 is a flat sheet comprising polyimide membranes of the present invention.

### DESCRIPTION OF THE INVENTION

The present invention generally relates to copolyimide membranes with unusually high selectivity and permeability for olefin/paraffin separations such as for ethylene/ethane, propylene/propane, butene/butane, and pentene/pentane separations and methods for making and using these membranes.

The present invention provides high selectivity and high permeability copolyimide membranes for olefin/paraffin separations. One copolyimide polymer used for the preparation of the high selectivity and high permeability polyimide membrane for olefin/paraffin separations in the present invention is a poly(3,3′-diaminodiphenyl sulfone-3,3′,5,5′-tetramethyl-4,4′-methylene dianiline-pyromellitic dianhydride) derived from the polycondensation reaction of 3,3′-diaminodiphenyl sulfone (3,3′-DDS) and 3,3′,5,5′-tetramethyl-4,4′-methylene dianiline (TMMDA) with pyromellitic dianhydride (PMDA). The molar ratio of 3,3′-DDS to TMMDA can be in a range from 10:1 to 1:10. Preferably, the molar ratio of 3,3′-DDS to TMMDA is in a range from about 5:1 to 1:5. The polyimide membrane described in the present invention is fabricated from the corresponding polyimide described herein. As an example, copolyimide membranes prepared from poly(3,3′-DDS-TMMDA-PMDA) with varying molar ratios of 3,3′-DDS to TMMDA (abbreviated as PI-DDS-T-1, PI-DDS-T-2, and PI-DDS-T-3) showed excellent separation properties for propylene/propane separation. The PI-DDS-T-1 membrane has shown high propylene permeability of 9.94 Barrers and high propylene/propane selectivity of 32.4 for propylene/propane separation. As another example, the PI-DDS-T-2 copolyimide membrane showed high propylene permeability of 6.24 Barrers and high propylene/propane selectivity of 28.1 for propylene/propane separation and the PI-DDS-T-3 copolyimide membrane showed even higher propylene permeability of 18.7 Barrers and high propylene/propane selectivity of 19.2.

Another copolyimide membrane described in the present invention is a poly(3,3′-DDS-TMMDA-3,5-diaminobenzoic acid-PMDA) derived from the polycondensation reaction of 3,3′-DDS, TMMDA, and 3,5-diaminobenzoic acid (DBA) with PMDA. The molar ratio of 3,3′-DDS to TMMDA is in a range from 10:1 to 1:10. The molar ratio of 3,3′-DDS to DBA is in a range from 10:1 to 1:10. The copolyimide membrane described in the present invention is fabricated from the corresponding copolyimide described herein. As an example, a copolyimide membrane prepared from poly(3,3′-DDS-TMMDA-DBA-PMDA) (abbreviated as PI-DDS-T-DBA-1) has shown good separation properties for propylene/propane separation. The PI-DDS-T-DBA-1 membrane showed propylene permeability of 4.98 Barrers and high propylene/propane selectivity of 19.1 for propylene/propane separation.

Yet another copolyimide membrane described in the present invention is a poly(3,3′-DDS-2,4,6-trimethyl-m-phenylenediamine-PMDA) derived from the polycondensation reaction of 3,3′-DDS and 2,4,6-trimethyl-m-phenylenediamine (TMPDA) with PMDA. The molar ratio of 3,3′-DDS to TMPDA is in a range from 10:1 to 1:10. As an example, a copolyimide membranes prepared from poly(3,3′-DDS-TMPDA-PMDA) (abbreviated as PI-DDS-TM-1) showed good separation properties for propylene/propane separation. The PI-DDS-TM-1 membrane has propylene permeability of 3.88 Barrers and high propylene/propane selectivity of 28.1 for propylene/propane separation.

The copolyimide with unusual high selectivity and permeability for the preparation of polyimide membrane for olefin/paraffin separations described in the present invention comprises a plurality of repeating units of formula (I).

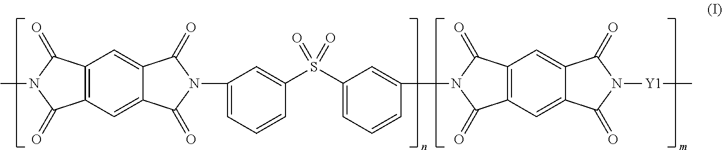

wherein m and n are independent integers from 10 to 500; wherein n/m is in a range of 10:1 to 1:10; wherein Y1 is selected from a group consisting of

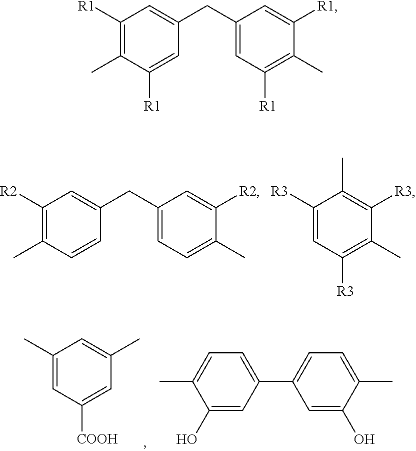

and mixtures thereof; wherein R1 is —(CH₂)ₚCH₃; wherein p is an integer from 0 to 11, preferably p is an integer of 0 or 1; wherein R2 is —(CH₂)_{q}CH₃; wherein q is an integer from 0 to 11, preferably q is an integer of 0 or 1; wherein R3 is —(CH₂)ᵣCH₃; wherein r is an integer from 0 to 11, preferably r is an integer of 0 or 1.

The copolyimide polymers shown in formula (I) used for making the copolyimide membranes with unusual high selectivity and high permeability for olefin/paraffin separations in the current invention have a weight average molecular weight in the range of 20,000 to 1,000,000 Daltons, preferably between 50,000 to 500,000 Daltons.

The copolyimide polymers shown in formula (I) described in the current invention can be synthesized from polycondensation reaction of a mixture of 3,3′-DDS and TMMDA with PMDA. The molar ratio of 3,3′-DDS to TMMDA is in a range of 10:1 to 1:10.

The solvents used for dissolving the copolyimide polymer with unusual high selectivity and permeability for the preparation of copolyimide membrane for olefin/paraffin separations are chosen primarily for their ability to completely dissolve the materials and for ease of solvent removal in the membrane formation steps. Other considerations in the selection of solvents include low toxicity, low corrosive activity, low environmental hazard potential, availability and cost. Representative solvents for use in this invention include, but are not limited to, N-methylpyrrolidone (NMP), N,N-dimethyl acetamide (DMAC), tetrahydrofuran (THF), acetone, N,N-dimethylformamide (DMF), dimethyl sulfoxide (DMSO), 1,3-dioxolane, and mixtures thereof. Other solvents as known to those skilled in the art may also be used.

The invention provides a process for separating olefins from a mixture of olefins and paraffins using the copolyimide membrane with unusually high selectivity and high permeability described in the present invention, the process comprising: (a) providing a copolyimide membrane with unusual high selectivity and high permeability described in the present invention which is permeable to said olefin; (b) contacting the olefin/paraffin mixture on one side of the copolyimide membrane with unusually high selectivity and high permeability described in the present invention to cause the olefin to permeate the membrane; and (c) removing from the opposite side of the membrane a permeate gas composition comprising a portion of the olefin which permeated through the membrane.

The copolyimide membrane with unusually high selectivity and high permeability described in the present invention has immediate application to concentrate olefin in a paraffin/olefin stream for an olefin cracking application. For example, the copolyimide membrane with unusually high selectivity and high permeability described in the present invention can be used for propylene/propane separation to increase the concentration of the effluent in a catalytic dehydrogenation reaction for the production of propylene from propane and isobutylene from isobutane. Therefore, the number of stages of a propylene/propane splitter that is required to get polymer grade propylene can be reduced. Another application for the copolyimide membrane described in the present invention is for separating isoparaffins and normal paraffins in a light paraffin isomerization and in MaxEne™, a process licensed by UOP LLC (Des Plaines, Ill.) for enhancing the concentration of normal paraffin (n-paraffin) in the naphtha cracker feedstock, which can be then converted to ethylene.

The various embodiments of the present invention provide a process for the separation of paraffin and olefin, such as, for example, in gaseous streams produced from stream cracking, catalytic cracking, and the dehydration of paraffins. The process utilizes a copolyimide membrane with unusually high selectivity and high permeability that is highly permeable but also highly selective to olefins, thus permitting olefins to permeate the membrane at a much higher rate than the paraffins. The membrane can take a variety of forms suitable for a particular application. For example, the membrane can be in the form of a flat sheet, hollow tube or fiber, spiral wound, and the like. In this regard, various embodiments of the process contemplated herein can be used to replace C2 and C3 splitters, as hybrid membrane distillation units for olefin purification, for recovery of olefins from polypropylene vent streams or from fluid catalytic cracking (FCC) off-gas streams, or the like. The process can also be used for the production of polymer grade propylene, thus offering significant energy, capital, and operating cost savings compared to conventional distillation.

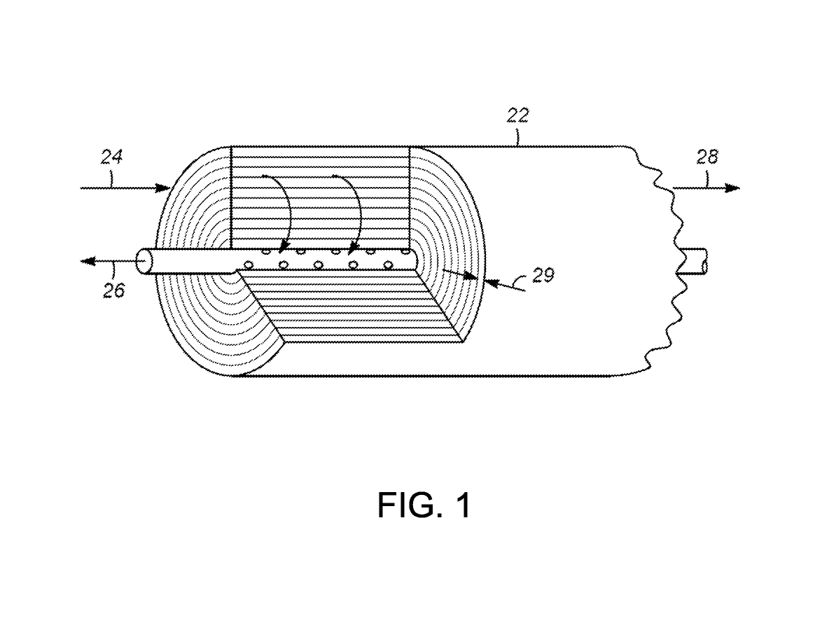

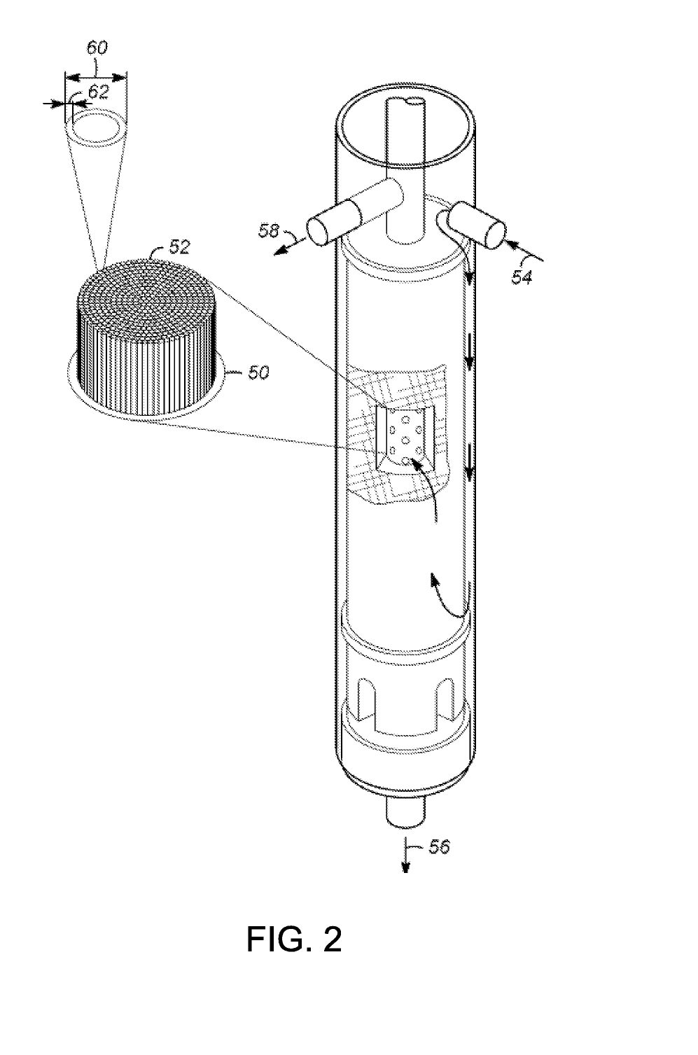

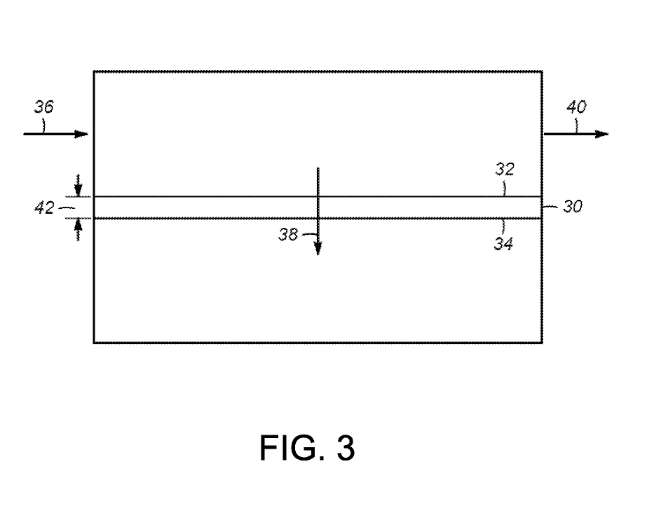

The copolyimide membranes with unusually high selectivity and high permeability for olefin/paraffin separations described in the present invention can be in any form suitable for a desired application. For example, the membranes can be in the form of hollow fibers or tubes, flat sheets, spiral wound, corrugated sheets, and the like. The form of the membrane may depend upon the nature of the membrane itself and the ease of manufacturing the form. The membrane can be assembled in a separator in any suitable configuration for the form of the membrane and the separator may provide for co-current, counter-current, or cross-current flows of the feed on the retentate and permeate sides of the membrane. In one exemplary embodiment, as illustrated in FIG. 1, a membrane 22 in a spiral wound module is in the form of flat sheet having a thickness 29 of from about 30 to about 400 μm. A feed 24 contacts a first surface of the membrane 22, a permeate 26 permeates the membrane 22 and is removed therefrom, and a retentate 28, not having permeated the membrane, also is removed therefrom. In another exemplary embodiment, as illustrated in FIG. 2, a membrane 50 in a hollow fiber module is in the form of thousands, tens of thousands, hundreds of thousands, or more, of parallel, closely-packed hollow fibers or tubes 52. In one embodiment, each fiber has an outside diameter 60 of from about 200 micrometers (μm) to about 700 millimeters (mm) and a wall thickness 62 of from about 30 to about 200 μm. In operation, a feed 54 contacts a first surface of the membrane 50, a permeate 56 permeates the membrane and is removed therefrom, and a retentate 58, not having permeated the membrane, also is removed therefrom. Referring to FIG. 3, a membrane 30 can be in the form of a flat sheet having a thickness 42 in the range of from about 30 to about 400 μm.

The olefin/paraffin separation process using the copolyimide membrane with unusually high selectivity and high permeability starts by contacting a first surface of the membrane with an olefin/paraffin feed. The olefin may comprise, for example, propylene or ethylene and the paraffin may comprise propane or ethane, respectively. The olefin/paraffin feed comprises a first concentration of olefin and a first concentration of paraffin depending on the application for which the membrane separation is used. The olefin/paraffin feed may comprise from about 5 to 95 mass % olefins and from about 5 to about 95 mass % paraffins. For example, a propane dehydrogenation process typically provides a feed containing about 35 mass % propylene whereas feed from an FCC unit generally contains about 75 mass % propylene. The flow rate and temperature of the olefin/paraffin feed have those values that are suitable for a desired application. Next, a permeate is caused to flow through the membrane and from a second surface of the membrane. Because the copolyimide membrane with unusually high selectivity and high permeability for olefin/paraffin separations is much more selective to the olefin than to the paraffin, the permeate has a concentration of olefin that is higher than the concentration of the olefin in the feed. In one exemplary embodiment, the concentration of the olefin in the permeate is 99.5 mass %. In addition, while some paraffin may permeate through the membrane, the permeate has a concentration of paraffin that is less than the concentration of the paraffin in the feed. The permeate can then be removed from the second surface of the membrane. As the permeate passes through the membrane, a retentate or residue, which has not permeated the membrane, is removed from the first surface of the membrane. The retentate has a concentration of olefin that is lower than the concentration of olefin in the feed and lower than the concentration of the permeate. The retentate also has a concentration of paraffin that is higher than a concentration of paraffin that is in the feed.

### EXAMPLES

The following examples are provided to illustrate one or more preferred embodiments of the invention, but are not limited embodiments thereof. Numerous variations can be made to the following examples that lie within the scope of the invention.

### Example 1

### Preparation and Evaluation of Copolyimide Dense Film Membranes from PI-DDS-T-1, PI-DDS-T-2 and PI-DDS-T-3 Polymers, Respectively

10.0 g of PI-DDS-T-1 (or PI-DDS-T-2 or PI-DDS-T-3) copolyimide synthesized from polycondensation reaction of a mixture of TMMDA and DDS diamines with PMDA dianhydride was dissolved in 40.0 g of NMP. The mixture was mechanically stirred for 2 hours to form a homogeneous casting dope. The resulting homogeneous casting dope was allowed to de-gas overnight. The PI-DDS-T-1 (or PI-DDS-T-2 or PI-DDS-T-3) dense film membrane was prepared from the bubble free casting dope on a clean glass plate using a doctor knife with a 18-mil gap. The membrane together with the glass plate was then put into a vacuum oven. The solvents were removed by slowly increasing the vacuum and the temperature of the vacuum oven. Finally, the PI-DDS-T-1, PI-DDS-T-2, and PI-DDS-T-3 copolyimide dense film membranes were heated at 200° C. under vacuum for 48 hours to completely remove the residual solvents.

The PI-DDS-T-1, PI-DDS-T-2 and PI-DDS-T-3 copolyimide dense film membranes were tested for propylene/propane separation at 50° C. under 791 kPa (100 psig) pure single feed gas pressure. The results in Table 2 show that PI-DDS-T-1, PI-DDS-T-2 and PI-DDS-T-3 copolyimide dense film membranes have excellent separation properties for propylene/propane separation. The PI-DDS-T-1 membrane has shown both high propylene permeability of 9.94 Barrers and high propylene/propane selectivity of 32.4. The PI-DDS-T-2 membrane has shown both high propylene permeability of 6.24 Barrers and high propylene/propane selectivity of 28.1 and the PI-DDS-T-3 membrane has shown even higher propylene permeability of 18.7 Barrers and a propylene/propane selectivity of 19.2.

TABLE 1
Pure gas permeation test results of PI-DDS-T-1, PI-DDS-T-2,
and PI-DDS-T-3 copolyimide dense film membranes
for propylene/propane separation*

| Membrane | P_{propylene} (Barrer) | α_{propylene/propane} |
|---|---|---|
| PI-DDS-T-1 | 9.94 | 32.4 |
| PI-DDS-T-2 | 6.24 | 28.1 |
| PI-DDS-T-3 | 18.7 | 19.2 |

*P_{propylene} and Pₚᵣₒₚₐₙₑ were tested at 50° C. and 791 kPa (100 psig);
1 Barrer = 10⁻¹⁰ cm³(STP) · cm/cm² · sec · cmHg.

### Example 2

### Preparation of Asymmetric Integrally-Skinned Flat Sheet PI-DDS-T-1 Copolyimide Membrane from PI-DDS-T-1 Copolyimide

An asymmetric integrally-skinned flat sheet PI-DDS-T-1 copolyimide membrane was prepared via a phase inversion process from a casting dope comprising, by approximate weight percentages, 10 g of PI-DDS-T-1 copolyimide, 35 g of N-methyl-2-pyrrolidone (NMP), 6.5 g of acetone, and 6.5 g of methanol. A film was cast on a Nylon cloth using a membrane casting machine then gelled by immersion in a 1.5° C. cold water bath, and then annealed in a hot water bath at 85° C. The resulting wet membrane was dried at 70° C. to remove water using a continuous membrane drier to form the dried flat sheet PI-DDS-T-1 copolyimide membrane. The dried membrane was then coated with a high permeance coating material to form the final asymmetric integrally-skinned flat sheet PI-DDS-T-1 copolyimide membrane of the present invention.

### Example 3

### Preparation of Asymmetric Integrally-Skinned Hollow Fiber PI-DDS-T-1 Copolyimide Membrane from PI-DDS-T-1 Polyimide

An asymmetric integrally-skinned hollow fiber PI-DDS-T-1 copolyimide membrane was prepared via a phase inversion process from a spinning dope comprising, by approximate weight percentages, 41 g of PI-DDS-T-1 polyimide, 70 g of NMP, 13 g of acetone, and 13 g of methanol. The spinning dope was extruded at a flow rate of 2.6 mL/min through a spinneret at 50° C. spinning temperature. A bore fluid containing 10% by weight of water in NMP was injected to the bore of the fiber at a flow rate of 0.8 mL/min simultaneously with the extruding of the spinning dope. The nascent fiber traveled through an air gap length of 5 cm at room temperature with a humidity of 25%, and then was immersed into a water coagulant bath at 21° C. and wound up at a rate of 8.0 m/min. The water-wet fiber was annealed in a hot water bath at 85° C. for 30 minutes. The annealed water-wet fiber was then sequentially exchanged with methanol and hexane for three times and for 30 minutes each time, followed by drying at 100° C. in an oven for 1 hour to form the dried hollow fiber PI-DDS-T-1 copolyimide membrane. The dried membrane was then coated with a high permeance coating material to form the final asymmetric integrally-skinned hollow fiber PI-DDS-T-1 copolyimide membrane of the present invention.

### Example 4

### Preparation and Evaluation of Copolyimide Dense Film Membranes from PI-DDS-T-DBA-1 and PI-DDS-TM-1

PI-DDS-T-DBA-1 and PI-DDS-TM-1 copolyimide dense film membranes were prepared from PI-DDS-T-DBA-1 and PI-DDS-TM-1 copolyimides, respectively, following the procedure described in Example 1.

The PI-DDS-T-DBA-1 and PI-DDS-TM-1 copolyimide dense film membranes were tested for propylene/propane separation at 50° C. under 791 kPa (100 psig) pure single feed gas pressure. The results in Table 3 show that PI-DDS-T-DBA-1 membrane showed propylene permeability of 4.98 Barrers and high propylene/propane selectivity of 19.1 for propylene/propane separation. The PI-DDS-TM-1 membrane has propylene permeability of 3.88 Barrers and high propylene/propane selectivity of 28.1 for propylene/propane separation.

TABLE 2
Pure gas permeation test results of PI-DDS-T-DBA-1 and PI-DDS-TM-1
copolyimide dense film membranes for propylene/propane separation*

| Membrane | P_{propylene} (Barrer) | α_{propylene/propane} |
|---|---|---|
| PI-DDS-T-DBA-1 | 4.98 | 19.1 |
| PI-DDS-TM-1 | 3.88 | 28.1 |

*P_{propylene} and Pₚᵣₒₚₐₙₑ were tested at 50° C. and 791 kPa (100 psig);
1 Barrer = 10⁻¹⁰ cm³(STP) · cm/cm² · sec · cmHg.

### Specific Embodiments

While the following is described in conjunction with specific embodiments, it will be understood that this description is intended to illustrate and not limit the scope of the preceding description and the appended claims.

A first embodiment of the invention is a copolyimide membrane comprising a plurality of repeating units of formula (I)

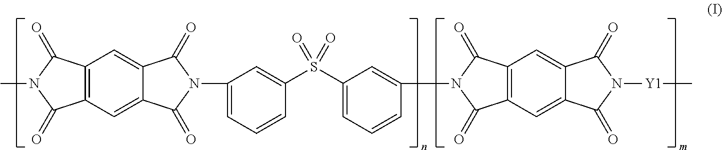

wherein m and n are independent integers from 10 to 500; wherein n/m is in a range of 101 to 110; wherein Y1 is selected from a group consisting of

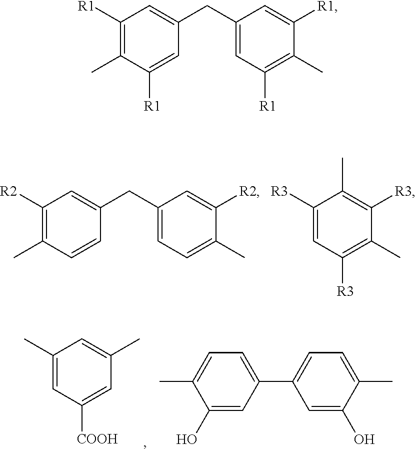

and mixtures thereof; wherein R1 is —(CH₂)ₚCH₃; wherein p is an integer from 0 to 11; wherein R2 is —(CH₂)_{q}CH₃; wherein q is an integer from 0 to 11; and wherein R3 is —(CH₂)ᵣCH₃; wherein r is an integer from 0 to 11. An embodiment of the invention is one, any or all of prior embodiments in this paragraph up through the first embodiment in this paragraph wherein the membrane comprises a poly(3,3′-diaminodiphenyl sulfone-3,3′,5,5′-tetramethyl-4,4′-methylene dianiline-pyromellitic dianhydride). An embodiment of the invention is one, any or all of prior embodiments in this paragraph up through the first embodiment in this paragraph wherein the membrane is in a form of hollow fibers, tubes, flat sheets, spiral wound modules or corrugated sheets.

A second embodiment of the invention is a process of preparing a poly(3,3′-diaminodiphenyl sulfone-3,3′,5,5′-tetramethyl-4,4′-methylene dianiline-pyromellitic dianhydride) membrane comprising reacting 3,3′-diaminodiphenyl sulfone and 3,3′,5,5′-tetramethyl-4,4′-methylene dianiline with pyromellitic dianhydride. An embodiment of the invention is one, any or all of prior embodiments in this paragraph up through the second embodiment in this paragraph wherein the 3,3′-diaminodiphenyl sulfone and the 3,3′,5,5′-tetramethyl-4,4′-methylene dianiline are present at a molar ratio in a range from about 101 to 110. An embodiment of the invention is one, any or all of prior embodiments in this paragraph up through the second embodiment in this paragraph wherein the 3,3′-diaminodiphenyl sulfone and the 3,3′,5,5′-tetramethyl-4,4′-methylene dianiline are present at a molar ratio in a range from about 51 to 15.

A third embodiment of the invention is a process for separating olefins from a mixture of olefins and paraffins comprising (a) providing a copolyimide membrane comprising a plurality of repeating units of formula (I)

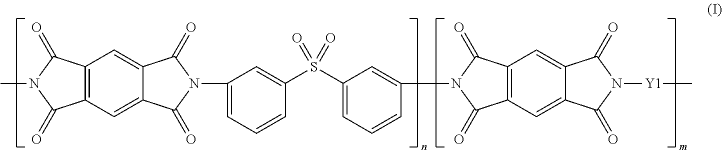

wherein m and n are independent integers from 10 to 500; wherein n/m is in a range of 101 to 110; wherein Y1 is selected from a group consisting of

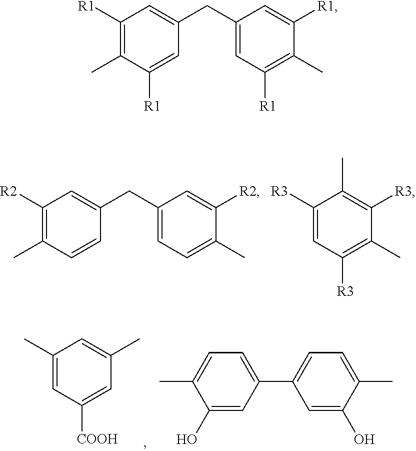

and mixtures thereof; wherein R1 is —(CH₂)ₚCH₃; wherein p is an integer from 0 to 11; wherein R2 is —(CH₂)_{q}CH₃; wherein q is an integer from 0 to 11; wherein R3 is —(CH₂)ᵣCH₃, and wherein r is an integer from 0 to 11; (b) contacting the olefin/paraffin mixture on one side of the copolyimide membrane to cause a portion of the olefins to permeate the membrane; and (c) removing from the opposite side of the membrane a permeate gas composition comprising the portion of the olefins which permeated through the membrane. An embodiment of the invention is one, any or all of prior embodiments in this paragraph up through the third embodiment in this paragraph wherein the olefins are selected from a group consisting of ethylene, propylene, butene, pentene or a mixture thereof. An embodiment of the invention is one, any or all of prior embodiments in this paragraph up through the third embodiment in this paragraph wherein the paraffins are selected from a group consisting of ethane, propane, butane, pentane or a mixture thereof. An embodiment of the invention is one, any or all of prior embodiments in this paragraph up through the third embodiment in this paragraph wherein the mixture of olefins and paraffins comprises from about 5-95 mass % olefin and about 5-95 mass % paraffins. An embodiment of the invention is one, any or all of prior embodiments in this paragraph up through the third embodiment in this paragraph wherein the portion of the olefins that permeated through the membrane comprises at least 80 mass % olefins. An embodiment of the invention is one, any or all of prior embodiments in this paragraph up through the third embodiment in this paragraph wherein the portion of the olefins which permeated through the membrane comprises a higher concentration of the olefins than the mixture of olefins and paraffins. An embodiment of the invention is one, any or all of prior embodiments in this paragraph up through the third embodiment in this paragraph wherein a retentate that has not penetrated the membrane comprises a higher concentration of the paraffins than the mixture of olefins and paraffins.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A copolyimide membrane comprising a plurality of repeating units of formula (I) 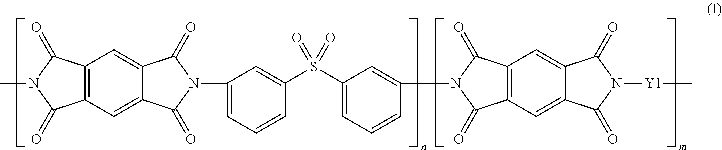
wherein m and n are independent integers from 10 to 500; wherein n/m is in a range of 10:1 to 1:10; wherein Y1 is selected from a group consisting of 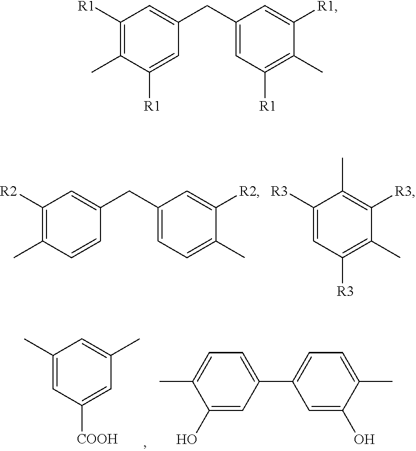
and mixtures thereof; wherein R1 is —(CH₂)ₚCH₃; wherein p is an integer from 0 to 11; wherein R2 is —(CH₂)_{q}CH₃; wherein q is an integer from 0 to 11; and wherein R3 is —(CH₂)ᵣCH₃; wherein r is an integer from 0 to 11.

2. The membrane of claim 1 comprising a poly(3,3′-diaminodiphenyl sulfone-3,3′,5,5′-tetramethyl-4,4′-methylene dianiline-pyromellitic dianhydride).

3. The membrane of claim 1 in a form of hollow fibers, tubes, flat sheets, spiral wound modules or corrugated sheets.

4. A process of preparing a poly(3,3′-diaminodiphenyl sulfone-3,3′,5,5′-tetramethyl-4,4′-methylene dianiline-pyromellitic dianhydride) membrane comprising reacting 3,3′-diaminodiphenyl sulfone and 3,3′,5,5′-tetramethyl-4,4′-methylene dianiline with pyromellitic dianhydride.

5. The process of claim 4 wherein said 3,3′-diaminodiphenyl sulfone and said 3,3′,5,5′-tetramethyl-4,4′-methylene dianiline are present at a molar ratio in a range from about 10:1 to 1:10.

6. The process of claim 4 wherein said 3,3′-diaminodiphenyl sulfone and said 3,3′,5,5′-tetramethyl-4,4′-methylene dianiline are present at a molar ratio in a range from about 5:1 to 1:5.

7. A process for separating olefins from a mixture of olefins and paraffins comprising:
(a) providing a copolyimide membrane comprising a plurality of repeating units of formula (I). 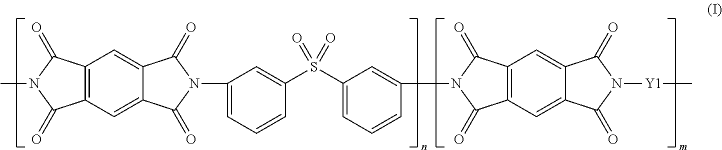
wherein m and n are independent integers from 10 to 500; wherein n/m is in a range of 10:1 to 1:10; wherein Y1 is selected from a group consisting of 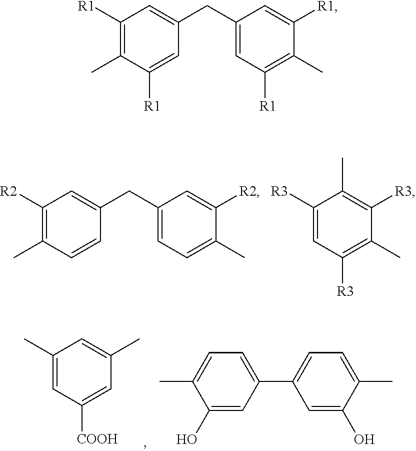
and mixtures thereof; wherein R1 is —(CH₂)ₚCH₃; wherein p is an integer from 0 to 11; wherein R2 is —(CH₂)_{q}CH₃; wherein q is an integer from 0 to 11; wherein R3 is —(CH₂)ᵣCH₃, and wherein r is an integer from 0 to 11;
(b) contacting the olefin/paraffin mixture on one side of the copolyimide membrane to cause a portion of the olefins to permeate the membrane; and
(c) removing from the opposite side of the membrane a permeate gas composition comprising the portion of the olefins which permeated through the membrane.

8. The process of claim 7 wherein said olefins are selected from a group consisting of ethylene, propylene, butene, pentene or a mixture thereof.

9. The process of claim 7 wherein said paraffins are selected from a group consisting of ethane, propane, butane, pentane or a mixture thereof.

10. The process of claim 7 wherein said mixture of olefins and paraffins comprises from about 5-95 mass % olefin and about 5-95 mass % paraffins.

11. The process of claim 7 wherein said portion of the olefins that permeated through the membrane comprises at least 80 mass % olefins.

12. The process of claim 7 wherein said portion of the olefins which permeated through said membrane comprises a higher concentration of said olefins than said mixture of olefins and paraffins.

13. The process of claim 7 wherein a retentate that has not penetrated said membrane comprises a higher concentration of said paraffins than said mixture of olefins and paraffins.
